(19) United States
(12) Patent Application Publication
(10) Pub. No.: US 2002/0165593 A1
(43) Pub. Date: Nov. 7, 2002

(54) Apparatus and method for aiding thrombosis through polymerization

(76) Inventors: Reid Hayashi, Palo Alto, CA (US); Mark T. Lemere, San Francisco, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/848,973

(22) Filed: May 3, 2001

(51) Int. Cl. A61N 1/00

(52) U.S. Cl. 607/88

(57) ABSTRACT

Disclosed is an apparatus and method for aiding processes including thrombosis, hemostasis, embolization, anastomotic sealing, and void filling at a treatment site of a patient. The present invention provides for an apparatus and method for polymerizing a material and introducing it to the treatment site. A material is polymerized by various methods, including the application of a catalyst, the application of ultraviolet light, or the application of electromagnetic radiation.

### BACKGROUND OF THE INVENTION

This invention relates to an apparatus and method for aiding thrombosis, and more particularly, to using polymerization for aiding processes including thrombosis, hemostasis, embolization, anastomotic sealing, and void filling at a treatment site of a patient.

Various blood vessels and organs are pierced or cut in connection with numerous surgical procedures or as a result of an accidental trauma. Some of these surgical procedures include percutaneous transluminal coronary angioplasty (PTCA) or angioplasty, angiography, biopsies, anastomosis procedures, as well as various neuro-interventional access procedures. These punctures or cuts may be life-threatening if not sealed.

Angiography is a diagnostic procedure wherein a dye is injected into an artery, preferably the femoral artery, to detect coronary disease. PTCA, or angioplasty, is a therapeutic procedure involving the inflation of a balloon in an artery, such as the coronary artery, for the purpose of clearing arterial occlusions. The femoral artery is incised, and a balloon catheter is inserted and fed to the treatment site in the coronary artery. The balloon is repeatedly inflated and deflated in an attempt to open the occlusion in the artery. Alternatively, a rotational tip catheter may also be used to remove plaque buildup utilizing a technique known as differential cutting or atheroctomy.

Angioplasty is more complicated and invasive than angiography, typically requiring the insertion of a larger sheath. The sheath is used to aid the introduction of the catheter into the artery. Angioplasty also requires the use of an anti-clotting agent, such as heparin, thus requiring a significant period of time to seal punctures or dissections.

During catheterization procedures, a physician or nurse will create an opening in an artery or other vessel using a conventional catheter introducer or dilator. Depending upon the type of procedure and the size of the catheter that is used, the size of the opening will vary. Additionally, a further enlargement of the incision or puncture will often occur as the catheter is twisted or otherwise manipulated while being advanced through the body of the patient.

One standard of care for puncture hemostasis has been applying manual pressure and pressure dressing on the puncture site until the puncture site is sealed by the natural coagulation of blood. Many of the patients undergoing these procedures have been medicated with an anticoagulant such as heparin, thus requiring a nurse to apply external pressure to the incision site for a lengthy period of time. This procedure may immobilize the patient for an extended period of time, resulting in great inconvenience, pain, anxiety, and discomfort for the patient, and a waste of time for both the medical personnel and the patient. Furthermore, the pressure application technique may fail to prevent hemorrhage and this may be life-threatening. Moreover, a painful hematoma or bruise may develop at the incision site because the vessel will continue to bleed internally until clotting blocks the opening in the vessel.

Consequently, there are specific instances where it is desirable to be able to deliver polymeric materials to a puncture site, open wound, or a surgical anastomosis using minimally invasive endoscopic or endovascular catheter technology to provide hemostasis and eventual healing within a patient's body. Numerous attempts have been made to aid hemostatic sealing of punctures and incisions in internal organs and blood vessels. Often these areas within the body are difficult to access.

Systems that have been employed in the past for occluding arteriovenous sites include pusher-vaso-occlusive coil assemblies and various embolic coils that were used in combination with catheters. The delivery of polymers has also been attempted. However, one problem associated with minimally invasive, endoscopic, or endovascular delivery of polymers is the viscosity of materials and the associated pressures required in order to deliver material through a small diameter lumen of a catheter.

References to thrombosis, defined as the coagulation of blood within a blood vessel, shall herein include thrombosis, hemostasis, embolization, anastomotic sealing, and void filling. “Hemostasis” refers to the stoppage of bleeding. “Embolization” refers to the occlusion of a blood vessel by a blood clot. “Anastomotic sealing” refers to the sealing of a surgical or traumatic opening between two normally distinct vessels. Finally, “void filling” refers to the filling of a hollow area, such as the space between an aneurysm sac and a therapeutically implanted graft, with a material to aid the coagulation of entrapped fluids.

What has been needed and heretofore unavailable is a relatively simplified, safe, fast-acting, and inexpensive process for aiding thrombosis. The present invention satisfies these needs.

### SUMMARY OF THE INVENTION

Briefly, and in general terms, the present invention is directed to an apparatus and method for aiding thrombosis at a treatment site of a patient. The present invention provides for an apparatus and method for polymerizing a material and introducing it to the treatment site. A material is polymerized by various methods, including the application of a catalyst, the application of ultraviolet light, or the application of electromagnetic energy.

In one presently preferred aspect, the invention is a method for aiding thrombosis at a treatment site of a patient. The method includes the steps of providing a material of low viscosity, providing a catalyst of low viscosity, and delivering the material and the catalyst to a treatment site of a patient. The material and the catalyst mix in vivo at the treatment site and polymerize.

In another aspect, the invention is a method for preventing blood from flowing around the outside of a graft. The method includes the steps of providing a first and a second material, and simultaneously delivering the first and second material into a perigraft space within a patient. Within the perigraft space, the first and second material mix in vivo and induce hemostasis.

In a further aspect, the invention is a system for aiding thrombosis at a treatment site of a patient. The system includes a catheter that has a proximal end and a distal end with a connector attached to the proximal-end. The connector has a primary port and a side port. An energy-curable polymer is injected into the side port. A polymer-curing energy source is included for administering energy into the proximal-end of the catheter. An energy guide having a proximal end and a distal end is longitudinally disposed within the catheter.

In yet another aspect, the invention is a method for aiding thrombosis at a treatment site of a patient. The method includes the steps of providing a polymeric material of low viscosity and delivering the material to a treatment site of a patient. The method also includes the step of polymerizing the material through the application of energy.

In a still further aspect, the invention is a method for aiding thrombosis at a treatment site of a patient. The method includes the steps of providing a polymeric material of low viscosity and delivering the material to a treatment site of a patient. Means for polymerizing the material is included, and the material is polymerized.

In another facet, the invention is a system for preventing blood from flowing around the outside of a graft. The system includes a catheter that has a proximal end and a distal end. A connector having a primary port and a side port is attached to the proximal-end of the catheter. An energy-curable polymer is provided for injection into the side port. A polymer-curing energy source is included for administering energy into the proximal-end of the catheter. An energy guide having a proximal end and a distal end is longitudinally disposed within the catheter.

In a further facet, the invention is a method for preventing blood from flowing around the outside of a graft. The method includes the steps of providing a polymeric material of low viscosity and delivering the material to a perigraft space within a patient. The material is polymerized through the application of energy.

In yet another facet, the invention is a method for preventing blood from flowing around the outside of a graft. The method includes the steps of providing a polymeric material of low viscosity and delivering the material to a perigraft space within a patient. Means for polymerizing the material is included, and the material is polymerized.

In a still further facet, the invention is a method for sealing an anastomotic leak. The method includes the step of providing a fluid and collagen particles. The fluid and collagen particles are delivered simultaneously for in vitro mixing, and dispensed at an anastomosis site, whereat the fluid-collagen mixture induces hemostasis.

Other features and advantages of the present invention will become more apparent from the following detailed description of the invention, when taken in conjunction with the accompanying exemplary drawings.

### BRIEF DESCRIPTION OF THE DRAWINGS

FIG. 1 is a schematic representation of the formulation of a two-part polymerized material mixture, wherein a catalyst is implemented;

FIG. 2a is a section view depicting a multi-lumen catheter for distributing the two materials of FIG. 1 for in vivo mixing;

FIG. 2b is a view depicting the catheter of FIG. 2 in use to deposit the two materials within the perigraft space of an abdominal aortic aneurysm;

FIG. 3 is a section view depicting the distal-end portion of a multi-lumen catheter used for in vitro mixing and distribution of a two-part material mixture;

FIG. 4 is a schematic of a device for curing a polymer string with an energy source and extruding it from a catheter;

FIG. 5a is a schematic of a device for curing a polymer string with an energy source and extruding it from a catheter;

FIG. 5b is a view depicting the device of FIG. 5a in use to deposit the polymer string within the perigraft space of an abdominal aortic aneurysm.

### DETAILED DESCRIPTION OF THE PREFERRED EMBODIMENTS

As shown in the exemplary drawings, the present invention is embodied in an apparatus and method for aiding thrombosis through the use of polymerization. Like reference numerals indicate like or corresponding elements among the figures.

As mentioned previously, organs and blood vessels may be pierced or cut in connection with various surgical procedures or as a result of an accidental trauma. Therefore, there are specific instances where it is desirable to be able to deliver polymeric materials to a puncture site, open wound, or a surgical anastomosis using minimally invasive endoscopic or endovascular catheter technology to provide hemostasis and eventual healing within a patient's body. Previous attempts have been made to aid hemostatic sealing of punctures and incisions in internal organs and blood vessels that are often difficult to access.

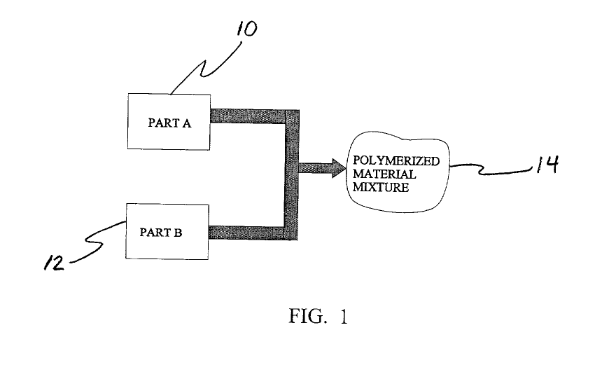

Referring to FIG. 1, in accordance with the present invention, materials are delivered to a treatment site of a patient in two parts or components, e.g., part A 10 and part B 12. The materials may be delivered via a multi-lumen catheter, two separate catheters, or another suitable device (not shown). Part A 10 is a polymer or copolymer mixture and part B 12 is a catalyst. In a preferred embodiment, part A 10 is a mixture of polyvinyl alcohol (PVA) and water, and part B 12 is a boric acid solution. In another embodiment, part B 12 includes gelatin particles suspended in the boric acid solution. Each component is of low viscosity, thereby allowing easy delivery through a small diameter lumen of a catheter. Upon mixing in vivo, part A 10 and part B 12 form a polymerized material mixture 14 that may be used for aiding processes including thrombosis at the treatment site of the patient. Thus, the wound is sealed and bleeding is stopped.

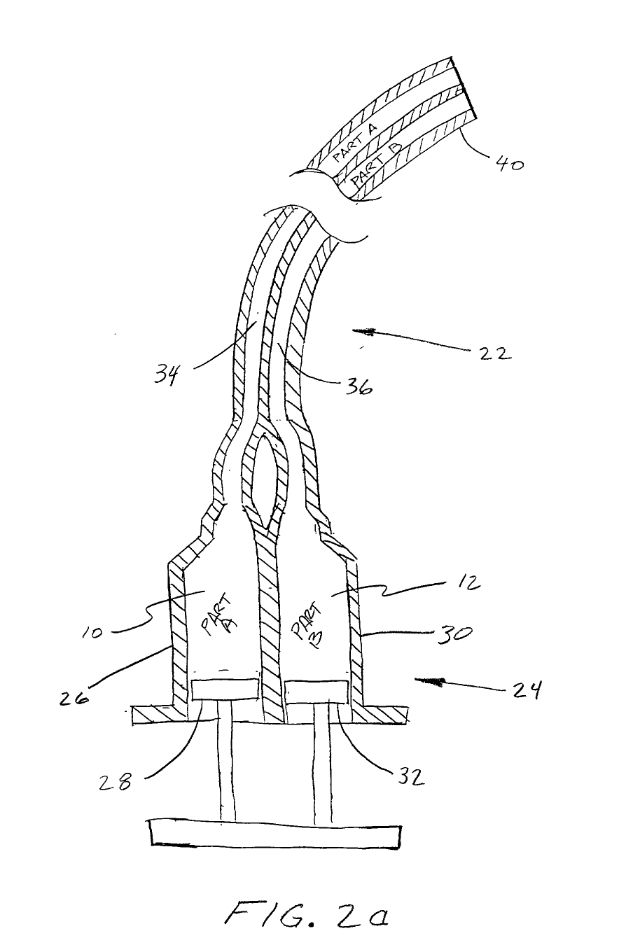

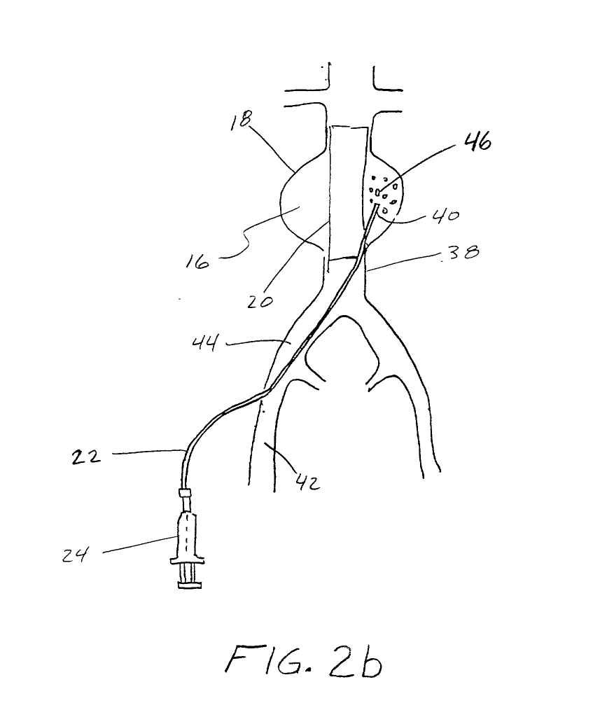

Referring to FIGS. 2a and 2b, part A 10 and part B 12 are delivered to a perigraft space 16 (the open void between an aneurysm, such as an aortic aneurysm 18, and an implanted graft 20) via a multi-lumen catheter, two separate catheters, or another suitable device in order to induce thrombosis and prevent blood from flowing around the outside of the graft. For demonstration purposes, a multi-lumen catheter 22 is shown in FIGS. 2a and 2b. In one embodiment, the catheter 22 includes a dual syringe 24 having a first tube 26 with a first plunger 28, and a second tube 30 with a second plunger 32. The catheter 22 also includes a first lumen 34 and a second lumen 36. At the proximal-end of the catheter 22, the first lumen 34 is attached to the distal-end of the first syringe tube 26, and the second lumen 36 is attached to the distal-end of the second syringe tube 30. Part A 10 is placed into the first tube 26 of the dual syringe 24 and part B 12 is placed into the second tube 30 of the dual syringe.

To distribute part A 10 and part B 12 into the perigraft space 16, such as within the abdominal aorta 38, the distal-end 40 of the catheter 22 is inserted into the femoral artery 42 and routed in the superior direction to the iliac artery 44, then into the abdominal aorta and the perigraft area. The distal-end 40 of the catheter 22 then pierces through the graft 20 and enters the perigraft space 16. The first 28 and second 32 plunger are then depressed into the first 26 and second 30 syringe tubes, thus propelling part A 10 and part B 12 into the first 34 and second 36 lumen respectively. As part A 10 and part B 12 simultaneously dispense from the distal-end 40 of the catheter 22, they mix in vivo and form a material mixture 46 that polymerizes within the perigraft space 16 and induces thrombosis. Thus, the flow of blood around the graft 20 is prevented.

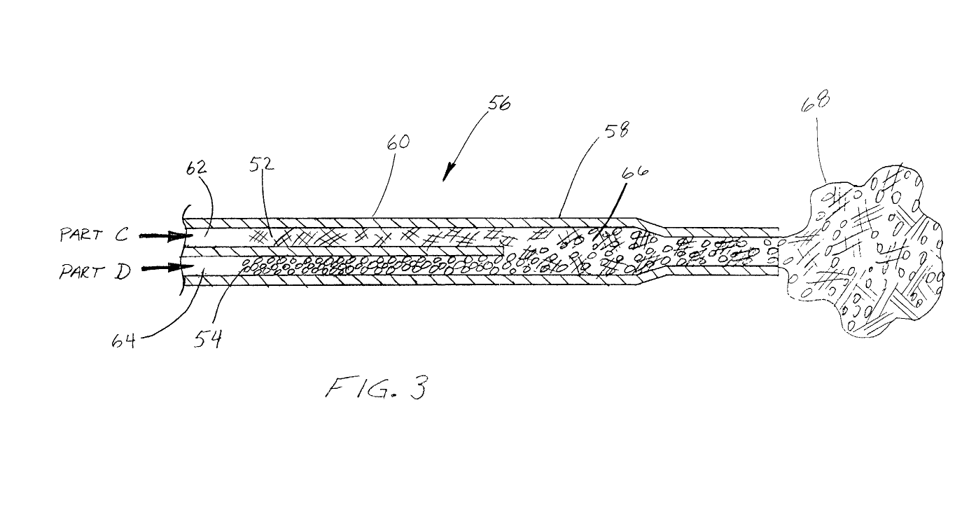

Referring to FIG. 3, materials are again delivered to a treatment site (not shown) of a patient in two parts, e.g., part C 52 and part D 54. In this embodiment, the materials are combined for the purpose of sealing anastomotic leaks. The materials may be delivered via a multi-lumen catheter, two separate catheters, or another suitable device. For demonstration purposes, a multi-lumen catheter 56 is shown in FIG. 3. Part C 52 is preferably a saline solution, plasma, or the patient's blood, and part D 54 is preferably dry collagen particles. Alternatively, part C 52 is hyaluronic acid, polyvinyl alcohol, or some other suitable liquid. Part C 52 is of low viscosity; thus allowing easy delivery through a small diameter lumen of a catheter. However, because part D 54 comprises dry particles, part D is propelled through the lumen by means such as hydraulic pressure, a push rod, or compressed gas such as carbon dioxide CO₂ (not shown). In one embodiment, part D 54 is a fibril form of collagen.

With continued reference to FIG. 3, at the distal-end region 58 of the catheter shaft 60, the first 62 and second 64 lumen merge to form a single lumen 66. As part C 52 and part D 54 are propelled from the first 62 and second 64 lumens, part C and part D enter the single lumen 66 and become mixed in vitro. The part C and part D mixture 68 then exits the single lumen 66, enters the region of the anastomosis, and induces hemostasis.

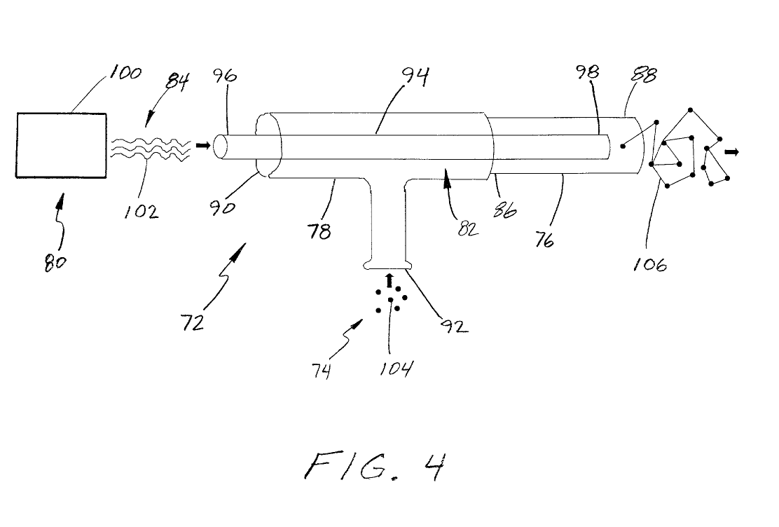

Referring to FIG. 4, an assembly 72 for curing and delivering an energy curable polymer 74 includes a catheter 76, a connector 78, a polymer curing energy source 80, and means 82 to deliver the polymer curing energy 84 to the polymer. In one preferred embodiment, the catheter 76 has a proximal end 86 and a distal end 88, and the connector 78 is attached to the proximal-end 86 of the catheter 76. The connector 78 has primary port 90 and side port 92. The means 82 for delivering the polymer-curing energy 84 is an optical fiber 94 that has a proximal end 96 and a distal end 98. The optical fiber 94 is inserted into the primary port 90 and is longitudinally disposed within the catheter 76. One type of an acceptable optical fiber 94 is comprised of acrylic. The polymer curing energy source 80 is preferably a tungsten-halogen lamp 100 that produces energy comprised of ultraviolet light 102. The distal-end 88 of the catheter 76 is subsequently delivered to a treatment site of a patient, whereupon the ultraviolet light 102 is sent from the proximal-end 96 of the optical fiber 94 to the distal-end 98 of the optical fiber. The curable polymer 74, or in this case, an ultraviolet-curable polymer 104, such as polyethylene glycol (PEG), is then injected into the side port 92. The polymer 104 then travels inside the catheter 76, reaches the distal-end 98 of the optical fiber 94, and comes into apposition with the ultraviolet light 102 whereby the polymer cures. The ultraviolet curable mixture consists of a monomer with a photoinitiator additive. Upon exposure to ultraviolet light, the radiation breaks the chemical bonds in the photoinitiator forming free radicals. The monomer component of the mixture then reacts with the free radicals and propagates as a free radical chain reaction, incorporating the monomers into a polymer chain. A polymer string 106, or cross-linked polymer, is subsequently extruded from the distal-end of the catheter 76, whereby the polymer string comes into apposition with the treatment site.

The above-recited steps do not necessarily have to occur in the stated order. For example, the step of delivering the catheter 76 may be performed before the step of inserting the optical fiber 94. Similarly, the step of injecting the ultraviolet curable polymer 104 may be performed before the step of sending the ultraviolet light 102. Consequently, the polymer string 106 may be used for aiding processes inducing thrombosis at a treatment site of a patient in a safe, easy, and efficient manner. Thus, the wound is sealed and bleeding is stopped.

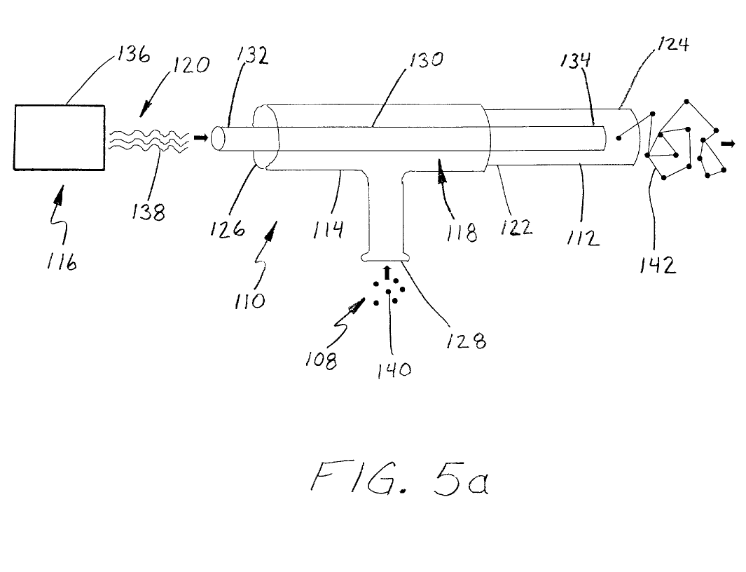

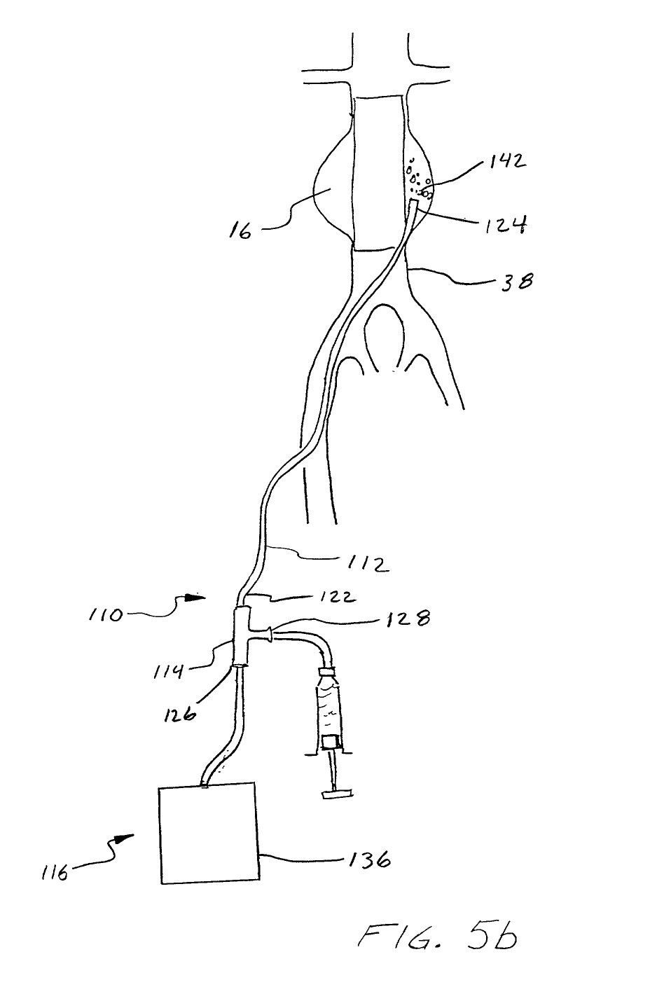

Referring to FIGS. 5a and 5b, an energy-curable polymer 108 is cured and delivered to a perigraft space 16 via an assembly 110 in order to induce thrombosis and prevent blood from flowing around the outside of the graft 20. The assembly 110 includes a catheter 112, a connector 114, a polymer curing energy source 116, and means 118 to deliver the polymer curing energy 120 to the polymer 108. In one preferred embodiment, the catheter 112 has a proximal end 122 and a distal end 124 and the connector 114 is attached to the proximal-end of the catheter. The connector 114 has a primary port 126 and a side port 128. The means 118 for delivering the polymer-curing energy 120 is an optical fiber 130 that has a proximal end 132, and a distal end 134. The optical fiber 130 is inserted into the primary port 126 and is longitudinally disposed within the catheter 112. One type of an acceptable optical fiber 130 is comprised of acrylic. The polymer curing energy source 116 is preferably an electromagnetic energy source 136 that produces energy comprised of electromagnetic radiation 138. The distal-end 124 of the catheter 112 is subsequently delivered to the perigraft space 16, such as within the abdominal aorta 38 via means as described above, whereupon the electromagnetic radiation 138 is sent from the proximal-end 132 of the optical fiber 130 to the distal-end 134 of the optical fiber. The curable polymer 108, or in this case, an electromagnetic radiation-curable polymer 140, such as polyethylene glycol (PEG), is then injected into the side port 128. The polymer 140 then travels inside the catheter 112, reaches the distal-end 134 of the optical fiber 130, and comes into apposition with the electromagnetic radiation 138 whereby the polymer cures. Alternatively, it is contemplated that the curable polymer 108 may be an ultraviolet-curable polymer and the polymer-curing energy source is an ultraviolet energy source. A polymer string 142, or cross-linked polymer, is thus extruded from the distal-end 124 of the catheter 112, whereby the polymer string is deposited within the perigraft space 16.

Again, the above-recited steps do not necessarily have to occur in the stated order. For example, the step of delivering the catheter 112 may be performed before the step of inserting the optical fiber 130. Similarly, the step of injecting the electromagnetic-curable polymer 140 may be performed before the step of sending the electromagnetic radiation 138. Consequently, the polymer string 142 may be used for aiding processes inducing thrombosis within the perigraft space 16 of a patient in a safe, easy, and efficient manner. Thus, the flow of blood around the graft 20 is prevented.

From the foregoing, it will be appreciated that the invention facilitates the inducement of thrombosis at a treatment site of a patient. The invention provides for an apparatus and method for polymerizing a material and introducing it to the treatment site. A material is polymerized by various methods, including the application of a catalyst, the application of ultraviolet light, or the application of electromagnetic radiation.

While the invention has been illustrated and described herein in terms of its use as an apparatus and method for aiding thrombosis, it will be apparent to those skilled in the art that the invention can be used in other instances. Other modifications and improvements may be made without departing from the scope of the invention.

What is claimed is:

1. A method for aiding processes including thrombosis, hemostasis, embolization, anastomotic sealing, and void filling at a treatment site of a patient through the in vivo mixing and polymerization of a low viscosity material and a low viscosity catalyst, comprising the steps of:
delivering the material to the treatment site of a patient; and
delivering the catalyst to the treatment site of a patient.

2. The method of claim 1, the material further comprising a polymer mixture.

3. The method of claim 1, the material further comprising a copolymer mixture.

4. The method of claim 1, the steps of delivering the material and the catalyst further comprising delivering the material and the catalyst via a dual-lumen system.

5. The method of claim 1:
the material further comprising polyvinyl alcohol mixed with water; and
the catalyst further comprising a boric acid solution.

6. The method of claim 1, the material further comprising polyvinyl alcohol mixed with water.

7. The method of claim 1, the catalyst further comprising gelatin particles suspended in a boric acid solution.

8. A method for preventing blood from flowing around the outside of a graft by inducing hemostasis via a first material and a second material, comprising the steps of:
delivering the first material and the second material simultaneously into a perigraft space within a patient; and
mixing the first material and the second material in vivo.

9. The method of claim 8, the step of delivering the first material and the second material further comprising delivering the first material and the second material via a dual-lumen system.

10. The method of claim 8:
the first material further comprising polyvinyl alcohol mixed with water; and
the second material further comprising a boric acid solution.

11. The method of claim 8, the first material further comprising polyvinyl alcohol mixed with water.

12. The method of claim 8, the second material further comprising gelatin particles suspended in a boric acid solution.

13. A system for aiding processes including thrombosis, hemostasis, embolization, anastomotic sealing, and void filling at a treatment site of a patient, comprising:
a catheter having a proximal end and a distal end;
a connector attached to the proximal-end of the catheter, the connector having a primary port and a side port;
an energy-curable polymer for injection into the side port;
a polymer-curing energy source for administering energy into the proximal-end of the catheter; and
an energy guide having a proximal end and a distal end, the energy guide being longitudinally disposed within the catheter.

14. The system of claim 13:
the polymer-curing energy source further comprising an ultraviolet light source;
the energy guide further comprising an optical fiber; and
the energy-curable polymer further comprising an ultraviolet-curable polymer.

15. A method for aiding processes including thrombosis, hemostasis, embolization, anastomotic sealing, and void filling at a treatment site of a patient via a low viscosity polymeric material, comprising the steps of:
delivering the material to a treatment site of a patient; and
applying energy to the material such that the material polymerizes.

16. The method of claim 15, the step of applying energy further comprising applying ultraviolet light.

17. A method for aiding processes including thrombosis, hemostasis, embolization, anastomotic sealing, and void filling at a treatment site of a patient via a low viscosity polymeric material and means for polymerizing the material, comprising the steps of:
delivering the material to a treatment site of a patient; and
polymerizing the material.

18. The method of claim 17, the means for polymerizing the material comprising ultraviolet light.

19. A system for preventing blood from flowing around the outside of a graft, comprising:
a catheter having a proximal end and a distal end;
a connector attached to the proximal-end of the catheter, the connector having a primary port and a side port;
an energy-curable polymer for injection into the side port;
a polymer-curing energy source for administering energy into the proximal-end of the catheter; and
an energy guide having a proximal end and a distal end, the energy guide being longitudinally disposed within the catheter.

20. The system of claim 19:
the polymer-curing energy source further comprising an ultraviolet light source;
the energy guide further comprising an optical fiber; and
the energy-curable polymer further comprising an ultraviolet-curable polymer.

21. The system of claim 19:
the polymer-curing energy source further comprising an electromagnetic radiation source; and
the energy-curable polymer further comprising an electromagnetic-curable polymer.

22. A method for preventing blood from flowing around the outside of a graft via a low viscosity polymeric material, comprising:
delivering the material to a perigraft space within a patient; and
applying energy to the material such that the material polymerizes.

23. The method of claim 22, the step of applying energy further comprising applying ultraviolet light.

24. The method of claim 22, the step of applying energy further comprising applying electromagnetic radiation.

25. A method for preventing blood from flowing around the outside of a graft via a low viscosity polymeric material and means to polymerize the material, comprising:
delivering the material to a perigraft space within a patient;
polymerizing the material.

26. The method of claim 25, the means for polymerizing the material comprising ultraviolet light.

27. The method of claim 25, the means for polymerizing the material comprising electromagnetic radiation.

28. A method for sealing anastomotic leaks via a fluid and collagen particles, comprising:
delivering the fluid and the collagen particles simultaneously to be mixed in vitro; and
dispensing the fluid and collagen particle mixture at an anastomosis site, whereupon the fluid and the collagen particle mixture induces hemostasis.

29. The method of claim 28, the step of delivering the fluid and collagen particles further comprising delivering the fluid and the collagen particles via a dual-lumen system.

30. The method of claim 28, wherein the fluid is selected from the group consisting of plasma, blood and saline.

31. The method of claim 28, the step of delivering the collagen particles further comprising propelling the collagen particles via hydraulic pressure.

32. The method of claim 28, the step of delivering the collagen particles further comprising propelling the collagen particles via a push rod.

33. The method of claim 28, the step of delivering the collagen particles further comprising propelling the collagen particles via compressed gas.
